(19) United States
(12) Patent Application Publication
(10) Pub. No.: US 2016/0081629 A1
(43) Pub. Date: Mar. 24, 2016

(54) METHOD AND DEVICE FOR DETERMINING A CORE BODY TEMPERATURE

(71) Applicant: Drägerwerk AG & CO. KGaA, Lübeck (DE)

(72) Inventors: Philipp ROSTALSKI, Lübeck (DE); Frank SATTLER, Lübeck (DE)

(73) Assignee: Drägerwerk AG & Co. KGaA, Lübeck (DE)

(21) Appl. No.: 14/888,555

(22) PCT Filed: Apr. 28, 2014
(86) PCT No.: PCT/EP2014/001124

§ 371 Date: Nov. 2, 2015

(30) Foreign Application Priority Data
May 2, 2013 (DE) 10 2013 007 631.5

(51) Int. Cl. A61B 5/00; A61B 5/01

(52) U.S. Cl. CPC A61B 5/7278; A61B 5/01; A61B 5/6801; A61B 2562/0271; A61B 2560/0223

(57) ABSTRACT

A method and a device for determine a core body temperature from a flow of heat from the body to a neutral medium via a first sensor element and a second sensor element. A dynamic model is used that describes heat flow with a plurality of parameters including the core body temperature, a first sensor element temperature, and a second sensor element temperature. The first sensor element is arranged on a surface of the body. One of the parameters and the core temperature are estimated such that a difference is minimized between the temperatures indicated by the sensor elements, and the temperatures resulting from the dynamic model at the first and second sensor elements for a plurality of time points which lie temporally prior to a specific time point. An estimated core temperature, where this difference has been minimized, is the core body temperature to be determined.

### CROSS REFERENCE TO RELATED APPLICATIONS

This application is a United States National Phase Application of International Application PCT/EP2014/001124 filed Apr. 28, 2014 and claims the benefit of priority under 35 U.S.C. §119 of German Patent Application 10 2013 007 631.5 filed May 2, 2013, the entire contents of which are incorporated herein by reference.

### FIELD OF THE INVENTION

The present invention pertains to a method for determining a core body temperature at a specific time from a heat flow from the body via a first sensor element and a second sensor element to a neutral medium, in which a dynamic model describing the heat flow with a plurality of parameters is used, wherein the model comprises, besides the plurality of the parameters describing the core body temperature, a temperature measured with the first sensor element and a temperature measured with the second sensor element, wherein the first sensor element is arranged on a surface of the body, wherein the second sensor element is arranged at such a spaced location from the first sensor element that a heat flow occurs between the first sensor element and the second sensor element as well as between the second sensor element and the neutral medium, and wherein the temperatures measured with the first and second sensor elements are recorded at a plurality of times before the specific time, as well as to a device for carrying out such a method.

### BACKGROUND OF THE INVENTION

Methods and devices for determining the inner temperature of a body, which is also called core temperature, are sufficiently known from the state of the art. The different approaches to measuring the core temperature or also body temperature can be classified at first, in principle, as invasive and non-invasive methods. Only non-invasive methods are used, if possible, in medicine, for example, in hospitals, and in safety engineering, because invasive methods are often not accepted by the persons to be monitored, they entail a certain risk of injury and are unsuitable for long-term monitoring of the core temperature.

One example of a non-invasive device for determining a core temperature is known from DE 100 38 247 A1, in which a so-called dual temperature sensor is disclosed. A dual temperature sensor has a first sensor element and a second sensor element in a common housing. The first sensor element is arranged on the surface of the body, for example, on the skin of a patient, and it measures a temperature there. The second sensor element is arranged in the housing, heat-insulated from the first sensor element, such that it measures a temperature in the housing. The temperatures measured by the sensor elements are transmitted to a data processing device connected to the sensor elements.

The data processing device uses a simple static model in order to determine the core temperature from the difference between the temperature measured with the first sensor element and the temperature measured with the second sensor element and two fixed coefficients of thermal conductivity. One of the coefficients of thermal conductivity, whose inverses are called thermal resistivities, describes the thermal conductivity of the material between the sensor elements and the other, the thermal conductivity of the body between the body core and the first sensor element. The coefficients of thermal conductivity form together with the core temperature and the temperature in the interior of the housing of the dual sensor, the parameters of the simple static model for the heat flow from the body via the first sensor element and the second sensor element to the surrounding area, whose variables are the temperatures measured with the first sensor element and the second sensor element.

The parameters that are used in the prior-art method to describe the thermal conductivity are implemented in the model with fixed values and, once the device is calibrated, are not adapted for the determination of the core temperature of different bodies or between two measurements of the core temperature on the same body. The coefficient of thermal conductivity for the material between the two sensors can be calibrated, for example, in a laboratory, and the coefficient of thermal conductivity between the body and the first sensor element is an empirical value.

Such devices have a great deal of drawbacks. First, the model does not take into account any heat capacities. If the prior-art dual sensor is placed on a body, the heating of the sensor elements is delayed because of the heat capacities of the sensor elements, the material between the sensor elements and the body itself. A reliable determination of the core temperature is thus possible, in principle, only if the dual sensor is at a thermal equilibrium. However, time periods between 10 minutes and 20 minutes may pass after placing a dual sensor until a thermal equilibrium becomes established.

In addition, a systematic error occurs, because the dual sensor uses the same coefficient of thermal conductivity for all bodies or patients. However, the coefficient of thermal conductivity, which shall describe the heat flow between the core of the body and the first sensor element, is subject to marked variations between different patients and also depends, last but not least, on how good the thermal coupling is between the sensor element and the surface of the body. However, the coupling depends, for example, on the nature of the surface and the contact between the sensor element and the surface.

WO 1998/050766 A1 describes an improved method for determining a core temperature, in which the dynamics of the heating of a dual sensor is taken at least partially into account. Instead of a static model for the thermal conductivity, the method is based on a partial differential equation, which has been approximately linearized by means of a plurality of hypotheses. The temperatures measured with the sensor elements are recorded by a data processing device at a plurality of times, for example, over several seconds. The core temperature and two parameters of the model, which describe the heat input into the first sensor element and the heat loss from the second sensor element, are estimated on the basis of the recorded temperatures. The model does not use any parameters that characterize the heat flow and that must or can be calibrated before a measurement. However, it was found in practice that the method has a low accuracy. In addition, it is not certain that the estimation of the core temperature and of the parameters converges, so that there is a possibility that the estimation yields false values for the core temperature.

### SUMMARY OF THE INVENTION

Therefore, an object of the present invention is to provide a method and a device which avoids the drawbacks known from the state of the art and makes it possible to determine a core temperature of a body rapidly and accurately.

In a first aspect of the present invention, the object is accomplished by a method in which at least one of the parameters and the core temperature are estimated at the specific time such that differences between the recorded temperatures and the temperatures that are obtained from the dynamic model for the plurality of times chronologically before the specific time is minimized. An estimated core temperature, at which the differences have been minimized, is determined and selected as the core body temperature to be determined.

The method according to the present invention is based, just as the methods known from the state of the art, on a mathematical representation of a heat flow from a body via a first sensor element and a second sensor element to a neutral medium. The term heat flow or even heat transport implies no special direction of the heat flow between two locations, but it only describes that two locations are thermally coupled. The term heat flow also does not point to an especially good thermal coupling of two objects. Thus, the first sensor element is in good thermal contact with the surface of the body, i.e., the thermal coupling is especially good between the surface of the body, for example, the skin of a patient, and the first sensor element in an exemplary embodiment. Even though the first sensor element and the second sensor element are arranged in a housing as a dual sensor in the exemplary embodiment, they are thermally insulated from one another. For example, the first sensor element and the second sensor element are separated from each other by an air gap. The thermal coupling between the first sensor element and the second sensor element is consequently only weak. By contrast, the second sensor element is in direct thermal contact with the neutral medium in the exemplary embodiment, so that there is a good thermal coupling here.

In an exemplary embodiment, the neutral medium is a test piece, which is heated by a heating device to a predetermined temperature. For example, the heat flow from the first sensor element to the second sensor element can thus be compensated, or the build-up time can be reduced. A preferred, alternative neutral medium is the area surrounding the body, whose core temperature shall be determined. for example, the air of the room in which a patient is located, or the interior of a housing of a dual sensor.

The first sensor element is arranged such that it is directly in contact with the surface of the body. For example, the sensor element—or a contact surface of the sensor element—lies flat on the skin of a patient, whose temperature shall be measured. The second sensor element is arranged at such a spaced location from the first sensor element that a heat flow occurs between the first sensor element and the second sensor element as well as between the second sensor element and the neutral medium. As was just already explained above, the two sensor elements are preferably arranged for this in a dual sensor, with the second sensor element or a contact surface of the second sensor element extending in parallel to the contact surface of the first sensor element and thus also points away from the surface of the body.

The temperatures measured with the first sensor element and the second sensor element are recorded. The sensor elements may be connected for this, for example, via a common data line, for example, to a data processing device, which comprises a storage medium, on which the temperatures or the measured values of the sensor elements can be stored. The data processing device may be arranged separated in space from the sensor elements or else even in a common housing with the sensor elements. The other steps of the method are preferably also carried out with the data processing device, which stores the data. The recording is performed at a plurality of times before a specific time, at which a core temperature shall be determined, for example, continuously every 200 msec.

The heat flow from the body through the sensor elements to the neutral medium is described with a dynamic model in this method. Different differential equations, which describe the heat transport with different accuracies, are sufficiently known to the person skilled in the art. Contrary to a static model, a dynamic model also describes time delays, which arise from the design of the measuring system. For example, heat capacities can be taken into account. With a few exceptions, a dynamic model does not describe a state of equilibrium, but a system, which develops from an initial state over time towards a state of equilibrium. For example, so-called build-up effects are thus taken into account.

The dynamic model or the differential equation, which describes the heat transport as a function of time, comprises at least one parameter, which describes or characterizes the heat transport. In addition, the model comprises at least the core temperature of the body and the temperatures measured at the two sensor elements. In an exemplary embodiment, the model also comprises the temperature of the neutral medium. However, it is also conceivable that the method is carried out with the use of additional sensor elements and the temperatures measured with these sensor elements are likewise included in the model as variables.

For example, the thermal conductivity of a component of a sensor element, a heat capacity of a component or, for example, of a surface of the body may be considered to be the at least one parameter. The model may comprise such parameters that had already been determined in advance in a calibrating measurement. However, it may also comprise other parameters that cannot be determined in a calibrating measurement. For example, these parameters depend on the body whose core temperature shall be determined, or on the thermal coupling between the surface of the body and the first sensor element.

To estimate the core temperature at the specific time, the at least one parameter and the core temperature are estimated. In addition, the temperature of the neutral medium is estimated in one exemplary embodiment. The estimation or even optimization of the parameters is carried out such that the difference between the temperatures that were measured with the first sensor element and the second sensor element and then recorded and the temperatures that the dynamic model predicts as a function of the at least one estimated parameter and the estimated core temperature is minimized. In other words, at least one parameter of the model and the core temperature are optimized in the model to the effect that the dynamic model predicts the course of the recorded temperatures with sufficient accuracy. In addition, the temperatures are predicted in one exemplary embodiment by the model as a function of an estimated temperature of the neutral medium.

The term “minimize” in this case does not mean that a minimum of the difference must actually be found. The parameters or temperatures are estimated or optimized only to the extent that the recorded temperatures are obtained from the dynamic model with sufficient accuracy.

The core temperature obtained by the estimation, in which the difference is minimized, represents the core body temperature to be determined. In other words, the value at which the difference between the temperatures predicted with the model and the measured temperatures is minimized or is sufficiently low is outputted as the core temperature that should be determined. This core temperature may be outputted, for example, via a display unit, which is connected to a data processing device carrying out the method.

The method according to the present invention makes it possible, compared to the methods known from the state of the art, to determine the core temperature of a body in a shorter time with higher accuracy, because the dynamic model takes build-up processes into account. The use of a dynamic model reduces the error that always occurs in static models unless the measuring system is at a thermal equilibrium.

Furthermore, at least one parameter of the model is estimated or optimized in the method according to the present invention. As a result, the method is flexibly adapted to changing measurement situations. For example, a parameter, which describes the heat transport from the body to the first sensor element, can be estimated. This parameter varies, for example, from one patient to another, and the thermal coupling may change continuously even in the same patient. Systematic errors, which occur due to the fixed preselection of such parameters, are avoided by the estimation.

In addition, the results determined with the method are markedly more accurate, because the dynamic model, which describes the heat transport, is used to determine or estimate the core temperature, and no simplification or linearization is required.

In a preferred embodiment, the plurality of parameters of the model comprise at least one parameter, which is a coefficient of thermal conductivity. The plurality of parameters preferably comprise at least one parameter, which describes in the model the heat flow from the body to the first sensor element in the form of a coefficient of thermal conductivity. Furthermore, it is preferable for the plurality of parameters to comprise at least one parameter that describes in the model the heat flow from the first sensor element to the second sensor element in the form of a coefficient of thermal conductivity, and/or for the plurality of parameters to comprise at least one parameter that describes in the model the heat flow from the second sensor element to the neutral medium in the form of a coefficient of thermal conductivity. The heat transport between the body and the first sensor element, between the first sensor element and the second sensor element and between the second sensor element and the neutral; medium is described by at least one coefficient of thermal conductivity each in a preferred exemplary embodiment.

In a preferred exemplary embodiment, the dynamic model may also comprise additional parameters, which are coefficients of thermal conductivity. For example, the heat transport from the body to the first sensor element may be described by two coefficients of thermal conductivity, one of which describes the heat transport through the body and the surface of the body and the other, the heat transport through the surface of a sensor housing, which extends between the sensor element and the surface of the body. It is not only a more accurate description of the heat transport that is made possible hereby. The parameter that describes the heat flow through the surface of the sensor housing can, moreover, be advantageously calibrated in laboratory measurements. The dynamic model thus has a more accurate resolution, even though the same number of parameters must be estimated.

In a preferred embodiment, the plurality of parameters of the model comprise at least one parameter that describes in the model a heat capacity. The plurality of parameters preferably comprise at least one parameter that describes in the model the heat flow from the body to the first sensor element in the form of a heat capacity. It is likewise preferred that the plurality of parameters comprise at least one parameter that describes in the model the heat flow from the first sensor element to the second sensor element in the form of a heat capacity, and/or that the plurality of parameters comprise at least one parameter that describes in the model the heat flow from the second sensor element to the neutral medium in the form of a heat capacity.

In a preferred exemplary embodiment, the model may comprise additional parameters, which describe a heat capacity, and the same explanations that were already given for additional coefficients of thermal conductivity apply to these parameters.

Furthermore, it is preferred to determine at least one of the parameters of the model in a calibrating measurement, and at least such parameters of the plurality of parameters are preferably determined in a calibration measurement that describe a heat flow from the first sensor element to the second sensor element. The heat flow from the first sensor element to the second sensor element is described, as a rule, with fixed, once calibrated values, because the sensor elements and the media between the sensor elements form a fixed system, which is independent from the body and the neutral medium. The parameters can therefore be determined accurately in the laboratory under laboratory conditions and be included in the model. The accuracy of the model and the time in which the core temperature can be determined with sufficient accuracy can be improved by the calibration of these parameters. Other parameters, which are independent from the body, may also be calibrated in other exemplary embodiments. Thus, at least one parameter, which describes the heat flow from the second sensor element to the neutral medium, is determined in the calibrating measurement in a preferred embodiment.

Such parameters of the plurality of parameters that describe a heat flow from the body to the first sensor element in the model are estimated in a preferred embodiment. These parameters depend on the body, the surface of the body, the contact between the surface and the first sensor element and many other variables, which vary not only from one body top another, but may also change during consecutive determinations of the core temperature of the same body. If the parameters are estimated rather than determined in advance, a systematic error is avoided in the determination of the core temperature.

In a preferred embodiment of the method, the core temperature to be determined is only outputted at the specific time if the difference between the temperature measured with the first sensor element at the specific time and the temperature that is obtained from the dynamic model with the estimated parameters for the specific time at the first sensor element falls below a preset value. It is likewise preferred to only output the core temperature to be determined at the specific time if the difference between the temperature measured with the second sensor element at the specific time and the temperature that is obtained from the dynamic model with the estimated parameters for the specific time at the second sensor element falls below a preset value.

In other words, a core temperature at the specific time is only outputted if the temperature measured with the first and/or second sensor element can be predicted with sufficient accuracy from the estimated parameters and the estimated core temperature. The accuracy of the predicted core temperature can also be especially advantageously predicted from the accuracy of the predicted temperatures at the first and/or second sensor element, and inaccurate values are no longer outputted. The difference may be, for example, the absolute or relative difference between two values. However, other measures with which the deviation between two values can be determined may also be used.

A quality factor, which is also outputted as a quality factor of the determined core temperature, is derived from the difference between the predicted and measured temperatures at the first and/or second sensor element in a preferred exemplary embodiment.

In another aspect, the object is accomplished by a device for determining and outputting a core temperature of a body with a first sensor element to be arranged on a surface of the body, a second sensor element, which is arranged at such a spaced location from the first sensor element that a heat flow can occur between the first sensor element and the second sensor element as well as between the second sensor element and the neutral medium, and with a data processing device, which has an output unit and is connected to the first sensor element and the second sensor element, and which is set up to record the temperatures measured with the sensor elements. The data processing device is set up to carry out a method in accordance with one of the above claims, wherein a core temperature determined with the method can be outputted by means of the output unit.

A data processing device, which is set up to carry out a method, has not only the ports that are necessary for connecting the sensors. Software, with which the method according to the present invention can be carried out or which executes the method according to the present invention, is also installed on the data processing device. The device may be arranged in a single housing, but it is also conceivable that the sensors, the data processing device and the output unit are arranged in separate housings. The data processing device may be, for example, a computer or other data processing device such as a microcontroller.

The same advantages as those already mentioned for the method according to the present invention are also obtained for the device according to the present invention.

The present invention will be explained below on the basis of drawings, which show two exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of an exemplary embodiment of a device according to the present invention; and

FIG. 2 is a flow chart of an exemplary embodiment of a method according to the present invention.

### DESCRIPTION OF THE PREFERRED EMBODIMENTS

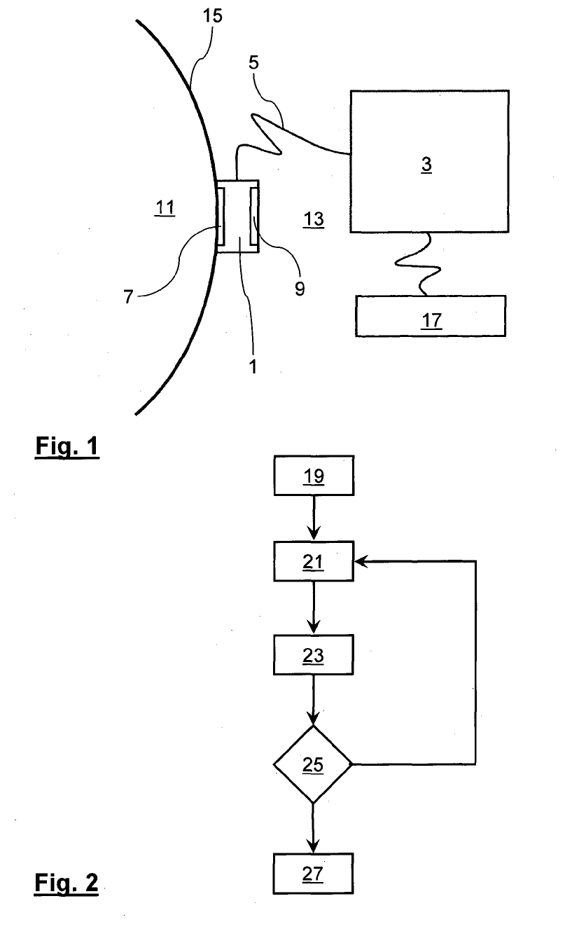

An exemplary embodiment of a device according to the present invention is first described with reference to FIG. 1. The device comprises a dual sensor 1 and a data processing device 3, which is connected to the dual sensor 1 via a data line 5. The dual sensor 1 comprises a first sensor element 7 and a second sensor element 9, which are arranged in a common housing and are connected to the data processing device 3 via the data line 5.

To carry out a method according to the present invention, the first sensor element 7 is placed flat on a body 11, whose core temperature shall be determined. The body 11, which is shown in FIG. 1 as a section only, is the body of a human being in the exemplary embodiment. However, the device according to the present invention may also be used to determine the core temperature of other bodies or objects.

The second sensor element 9, which is in thermal contact with a neutral medium 13, which is formed by the surrounding area 13 of the body 11 or by the air 13 around the body 11, is arranged on the opposite side of the housing of the dual sensor 1. The surrounding area 13 has an ambient temperature that changes only slowly.

To carry out a method according to the present invention, the first sensor element 7 and the second sensor element 9 are arranged such that a heat flow becomes established from the body 11 via a surface 15 of the body 11 through the dual sensor 1 to the neutral medium 13. The first sensor element 7 is in contact for this with the surface 15 of the body 11 and the first sensor element 7 is thermally coupled via the skin 15 with the body 11, whose core temperature shall be determined. The dual sensor 1 or the second sensor element 9 is arranged such that a heat flow becomes established from the first sensor element 7 via the second sensor element 9 to the neutral medium 13 or the surrounding area 13. In other words, the second sensor element 9 is coupled thermally with both the surrounding area 13 and with the first sensor element 7.

The first sensor element 7 and the second sensor element 9 measure a temperature at a plurality of times and preferably continuously and transmit the measured temperatures to the data processing device 3, which can record the temperatures at least for a plurality of times.

Based on the recorded temperatures, the data processing device 3 carries out a method according to the present invention, which will be described below. The data processing device 3 is set up (configured) to carry out the method. In other words, the data processing device 3 has connections via which the sensor elements 7, 9 can be connected to the data processing device 3. Corresponding software, which can carry out the method, is also provided or installed on the data processing device 3.

The core temperature determined with the method according to the present invention can be outputted via an output unit 17, which is connected to the data processing device 3. For example, the output unit 17 is a screen, on which a determined core temperature can be displayed. However, the determined core temperature may be outputted on paper by means of a printer.

In the exemplary embodiment, the dual sensor 1, the data processing device 3 and the output unit 17 are shown schematically in separate housings or as independent units. However, the dual sensor 1, the data processing device 3 and the output unit 17 may be arranged in a common housing.

An exemplary embodiment of a method according to the present invention for determining the core temperature is explained below with reference to the flow chart shown in FIG. 2, wherein the method is described as an example with reference to the exemplary embodiment of the device according to the present invention from FIG. 1. However, the exemplary embodiment of the method according to the present invention is not limited to the specific exemplary embodiment of the device.

To determine the core temperature, the dual sensor 1 and hence the first sensor element 7 are arranged in a first step on a surface 15 of the body 11, whose core temperature shall be determined. The second sensor element 9 thus points away from the surface 15 and is in thermal contact with the first sensor element 7 and the neutral medium 13.

To determine the temperature at a specific time, the temperatures measured by the first and second sensor elements 7, 9 at the plurality of times are recorded by the data processing device 3 in another step 21 at a plurality of times, which chronologically precede the specific time. Based on the recorded temperatures, the data processing device 3 then estimates different parameters of a dynamic model for a heat flow in a subsequent step 23.

The dynamic model describes the heat flow from the body 11 via the first and second sensor elements 7, 9 to the neutral medium 13. A dynamic model already differs from a static model in that a dynamic model does not describe a static state of equilibrium, but a system with latencies, which develops from a starting situation towards another state and preferably towards a state of equilibrium. In other words, a dynamic model changes over time, even if the other input variables or parameters do not change, until a state of equilibrium is reached.

The dynamic model first comprises a number of temperatures. These include the temperatures that are measured at or with the first and second sensor elements 7, 9, as well as the core temperature of the body 11. Furthermore, the dynamic model is formed by a number of parameters, which describe or characterize the heat flow from the body 11 through the dual sensor 1 or the first sensor element 7 and the second sensor element 9 towards the surrounding area.

In this exemplary embodiment, the model comprises the heat capacity and the thermal conductivity or even thermal resistivities of the section of the dual sensor 1 located between the first sensor element 7 and the second sensor element 9. Furthermore, the dynamic model comprises the heat capacity and the thermal conductivity of the section between a core of the body 11 and the first sensor element 7. The thermal conductivity of the second sensor element 9 is also included in the model. There is no strict separation between the parameters that are determined by the behavior between two locations at which the temperature is determined, e.g., the first sensor element 7 and the second sensor element 9, and the parameters that are described by the sensor elements 7, 9 themselves.

At least one of the parameters that describe the heat flow and the core temperature are estimated in a subsequent step 23 of the method such that a difference between the recorded temperatures and the temperatures that are obtained from the dynamic model at the sensor elements 7, 9 for the plurality of times is minimized. An optimization method, which varies the parameters until the difference has been minimized, is used for this. The term “minimized” does not mean that an absolute minimum has been found, but only that convergence criteria fixed in advance have been met, or only a fixed number of iterations is performed in the optimization method. The core temperature thus estimated may be outputted via the output unit 17.

Not all the parameters that describe the heat flow are estimated by the method in the exemplary embodiment of the method. Such parameters that are largely or fully independent from the body 11 proper, whose core temperature shall be determined, are determined or calibrated in calibrating measurements in the laboratory before the method is carried out. These include, for example, the thermal conductivity and the heat capacity of the section of the dual sensor 1 located between the first and second sensor elements 7, 9. The thermal conductivity between the second sensor element 9 and the neutral medium 13 may also be determined in a calibrating measurement.

By some of the parameters being fixed in advance in calibrating measurements, the number of parameters that must be estimated is reduced. As a result, the plurality of times are) reduced or the measurement time after which a sufficiently accurate core temperature can already be determined becomes shorter.

The other parameters, which cannot be determined in calibrating measurements in the laboratory, are estimated in the method. A systematic error, as it is known from the state of the art, is thus avoided. In particular, the parameters that describe or characterize the heat transport from the body 11 to the first sensor element 7 are estimated. These parameters change not only from one body to the next, but also between two measurements on the same body. A systematic error, which becomes established if fixed parameters are used, is avoided due to the estimation of the parameters.

Furthermore, a markedly faster convergence of the core temperature determination is obtained from the method according to the present invention, i.e., the method yields a more accurate core temperature already after a shorter measurement time than the methods known from the state of the art, because of the use of a dynamic method takes into account the build-up effects that occur before the system comprising the body 11, the first and second sensor elements 7, 9 and the neutral medium 13 is in a thermal equilibrium.

Before the core temperature is outputted in the exemplary embodiment of the method according to the present invention, a check is, however, made in an additional step 25 to determine whether the dynamic model predicts the temperature at the first and second sensor elements 7, 9 at the specific time at which the core temperature shall be outputted with sufficient accuracy, i.e., when the difference between temperatures predicted for the specific time and actually measured temperatures is sufficiently small. It is only when this is the case that the core temperature is outputted with the output unit 17 in a subsequent step 27. If the temperatures are not predicted with sufficient accuracy, the estimation of the parameters and the core temperature must be repeated over a changed time period or over a changed plurality of times.

The accuracy of the determined core temperature at the time of the output is thus checked in an especially advantageous manner. In addition, an additional criterion can thus be set to check whether the time period underlying the estimation of the parameters or the plurality of times was sufficiently large or whether the estimation carried out so far is still valid or a new estimation must be performed.

While specific embodiments of the invention have been shown and described in detail to illustrate the application of the principles of the invention, it will be understood that the invention may be embodied otherwise without departing from such principles.

1. A method for determining a core temperature of a body, at a specific time, from heat flow from the body via a first sensor element and a second sensor element to a neutral medium, the method comprising the steps of:
providing a dynamic model describing the heat flow with a plurality of parameters, wherein the model comprises at least the core temperature of the body, a temperature measured with the first sensor element and a temperature measured with the second sensor element, besides the plurality of parameters describing the heat flow;
arranging the first sensor element on a surface of the body;
arranging the second sensor element at a spaced location from the first sensor element such that heat flow occurs between the first sensor element and the second sensor element as well as between the second sensor element and the neutral medium;
recording temperatures measured with the first and second sensor elements at a plurality of times before the specific time;
estimating at least one of the parameters and the core temperature at the specific time such that differences between the recorded temperatures and the temperatures that are obtained from the dynamic model for the plurality of times located chronologically before the specific time at the first and second sensor elements are minimized; and
determining an estimated core temperature, at which the differences have been minimized, as the core temperature of the body that is to be determined.

2. A method in accordance with claim 1, wherein the plurality of parameters of the model comprise at least one parameter, which is a coefficient of thermal conductivity.

3. A method in accordance with claim 2, wherein at least one of:
the plurality of the parameters in the model comprise at least one parameter that describes the heat flow from the body to the first sensor element in the form of the coefficient of thermal conductivity;
the plurality of the parameters comprises at least one parameter that describes in the model the heat flow from the first sensor element to the second sensor element in the form of the coefficient of thermal conductivity; and
the plurality of the parameters comprises at least one parameter that describes in the model the heat flow from the second sensor element to the neutral medium in the form of a coefficient of thermal conductivity.

4. A method in accordance with claim 1, wherein the plurality of the parameters of the model comprise at least one parameter that describes a heat capacity in the model.

5. A method in accordance with claim 4, wherein:
the plurality of the parameters comprise at least one parameter that describes in the model the heat flow from the body to the first sensor element in the form of the heat capacity;
the plurality of the parameters comprise at least one parameter that describes in the model the heat flow from the first sensor element to the second sensor element in the form of the heat capacity; and
the plurality of the parameters comprise at least one parameter that describes in the model the heat flow from the second sensor element to the neutral medium in the form of the heat capacity.

6. A method in accordance with claim 1, wherein at least one of the parameters of the model is determined in a calibrating measurement.

7. A method in accordance with claim 6, wherein at least such parameters of the plurality of parameters that describe a heat flow from the first sensor element to the second sensor element are determined in a calibrating measurement.

8. A method in accordance with claim 6, wherein at least one parameter, which describes the heat flow from the second sensor element to the neutral medium, is determined in a calibrating measurement.

9. A method in accordance with claim 1, wherein at least such parameters of the plurality of parameters that describe in the model a heat flow from the body to the first sensor element are estimated.

10. A method in accordance with claim 1, wherein:
the core temperature to be determined at the specific time is only outputted if the difference between the temperature measured at the specific time with the first sensor element and the temperature that is obtained from the dynamic model with the estimated parameters for the specific time at the first sensor element falls below a preset value; and
the core temperature to be determined at the specific time is outputted only if the difference between the temperature measured at the specific time with the second sensor element and the temperature that is obtained from the dynamic model with the estimated parameters for the specific time at the second sensor element falls below a preset value.

11. A method in accordance with claim 1, wherein the neutral medium is an area surrounding the body.

12. A device for determining and outputting a core temperature of a body the device comprising:
a first sensor element to be arranged on a surface of the body;
a second sensor element, which is arranged at such a spaced location from the first sensor element that heat flow can occur between the first sensor element and the second sensor element as well as between the second sensor element and a neutral medium;
a data processing device, which has an output unit, the data processing device being connected to the first and second sensor elements and being set up to record temperatures measured with the sensor elements, wherein the data processing device is set up to provide a dynamic model describing the heat flow with a plurality of parameters, the model comprising at least the core temperature of the body, a temperature measured with the first sensor element and a temperature measured with the second sensor element, besides the plurality of parameters describing the heat flow, the data processing device being configured to execute the method comprising:
recording temperatures measured with the first and second sensor elements at a plurality of times before a specific time;
estimating at least one of the parameters and the core temperature at the specific time such that differences between the recorded temperatures measured with the first and second sensor elements at a plurality of times and the first and second sensor element temperature values that are obtained from the dynamic model, for the plurality of times chronologically before the specific time at the first and second sensor elements are minimized; and
determining the estimated core temperature, at which the differences have been minimized, to be the core temperature of the body that is to be determined, wherein a core temperature determined with the method is outputted by means of the output unit.

13. A device accordance with claim 12, wherein the plurality of parameters of the model comprise at least one parameter, which is a coefficient of thermal conductivity.

14. A device in accordance with claim 12, wherein the plurality of parameters in the model comprise:
at least one parameter that describes the heat flow from the body to the first sensor element in the form of a body to first sensor element coefficient of thermal conductivity;
at least one parameter that describes the heat flow from the first sensor element to the second sensor element in the form of a first sensor element to second sensor element coefficient of thermal conductivity;
at least one parameter that describes the heat flow from the second sensor element to the neutral medium in the form of a second sensor element to neutral medium coefficient of thermal conductivity.

15. A device in accordance with claim 12, wherein the plurality of the parameters of the model comprise at least one parameter that describes a heat capacity.

16. A device in accordance with claim 12, wherein the plurality of the parameters comprise:
at least one parameter that describes heat flow from the body to the first sensor element in the form of a body to the first sensor element heat capacity;
at least one parameter that describes heat flow from the first sensor element to the second sensor element in the form of a first sensor element to second sensor element heat capacity; and
at least one parameter that describes heat flow from the second sensor element to the neutral medium in the form of a second sensor element to neutral medium heat capacity.

17. A device in accordance with claim 12, wherein at least one of the parameters of the model is determined in a calibrating measurement of the device.

18. A device in accordance with claim 12, wherein at least one parameter, of the plurality of parameters, that describe heat flow from the first sensor element to the second sensor element are determined in a calibrating measurement of the device.

19. A device in accordance with claim 12, wherein at least one parameter, of the plurality of parameters, that describes heat flow from the second sensor element to the neutral medium, is determined in a calibrating measurement of the device.

20. A device in accordance with claim 12, wherein:
the neutral medium is an area surrounding the body;
the core temperature to be determined at the specific time is only outputted if the difference between the temperature measured at the specific time with the first sensor element and the temperature that is obtained from the dynamic model with the estimated parameters for the specific time at the first sensor element falls below a preset value; and
the core temperature to be determined at the specific time is outputted only if the difference between the temperature measured at the specific time with the second sensor element and the temperature that is obtained from the dynamic model with the estimated parameters for the specific time at the second sensor element falls below a preset value.

21. A method for determining a core temperature of a body in a neutral medium, the method comprising the steps of:
arranging a first sensor element on a surface of the body;
arranging a second sensor element at a spaced location from said first sensor element by a spacer material such that a heat flow from the body flows through said first sensor element, said spacer material, said second sensor element and to the neutral medium;
recording temperatures measured with said first and second sensor elements at a plurality of times during which the body, said sensor elements, said spacer material and the neutral medium are not in thermal equilibrium; and
using said temperatures to estimate the core temperature of the body.

22. A method in accordance with claim 21, wherein:
the body has a thermal conductivity and a heat capacity;
a dynamic model is provided representing the heat flow over time from the body through said first sensor element, said spacer material, said second sensor element and to the neutral medium, said dynamic model incorporating said thermal conductivity and said heat capacity of the body;
using said temperatures at said plurality of times in said dynamic model to estimate said thermal conductivity and said heat capacity of the body.

23. A method in accordance with claim 22, wherein:
said spacer material has a thermal conductivity and a heat capacity, said thermal conductivity and said heat capacity of said spacer material being predetermined;
said dynamic model incorporates said thermal conductivity and said heat capacity of said spacer material, said dynamic model represents the heat flow when said spacer material and the neutral medium are not in thermal equilibrium.
